(19) United States
(12) Patent Application Publication
(10) Pub. No.: US 2012/0329089 A1
(43) Pub. Date: Dec. 27, 2012

(54) METHODS OF GENERATING HYDROGEN

(76) Inventor: Jacob Edrei, Kiryat-Tivon (IL)

(21) Appl. No.: 13/582,442

(22) PCT Filed: Mar. 10, 2011
(86) PCT No.: PCT/IL11/00235

§ 371 Date: Sep. 3, 2012

Related U.S. Application Data
(60) Provisional application No. 61/312,678, filed on Mar. 11, 2010.

(51) Int. Cl. C12P 3/00; C12M 1/42; C12P 39/00

(52) U.S. Cl. 435/42; 435/292.1; 435/168

(57) ABSTRACT

The present invention, in some embodiments thereof, relates to a photocatalytic method of generating hydrogen gas in algae, and, more particularly, but not exclusively, to algal-bacterial co-culture for enhancing the kinetics and improving the yield of algal hydrogen photoproduction.

### RELATED APPLICATION/S

This application claims the benefit of priority under 35 USC 119(e) of U.S. Provisional Patent Application No. 61/312,678 filed Mar. 11, 2010, the contents of which are incorporated herein by reference in their entirety.

### FIELD AND BACKGROUND OF THE INVENTION

The present invention, in some embodiments thereof, relates to a photocatalytic method of generating hydrogen gas in algae.

Hydrogen gas (molecular hydrogen) is thought to be the ideal fuel for a world in which air pollution has been alleviated, global warming has been arrested, and the environment has been protected in an economically sustainable manner, since combustion of hydrogen gas liberates large amounts of energy per weight without producing CO₂ (produces H₂O instead) and hydrogen is easily converted to electricity by fuel cells. Hydrogen and electricity could team to provide attractive options in transportation and power generation. Interconversion between these two forms of energy suggests on-site utilization of hydrogen to generate electricity, with the electrical power grid serving in energy transportation, distribution utilization, and hydrogen regeneration as needed. However, the renewable and environmentally friendly generation of large quantities of H₂ gas poses a challenging problem for the use of H₂ as a source of energy for the future. Biological hydrogen production has several advantages over photoelectrochemical, or thermochemical processes, as it requires only simple solar reactors, with low energy requirements, in place of high energy-requiring batteries to power electrochemical processes.

Cyanobacteria and green algae are the only known organisms with both an oxygenic photosynthesis and hydrogen production. In the mid-1900s, hydrogen production was first observed in the green alga Scenedesmus, upon illumination after incubation in anaerobic and light-restricted conditions (dark adaptation). Since then, photobiological hydrogen gas production in green microalgae has attracted much attention, with the goal of utilizing the photosynthetic electron transport pathway as a source of electrons for reduction of H⁺ to hydrogen gas by the ferredoxin-linked hydrogenase pathway. In addition, under anaerobic conditions, fermentation of carbon compounds can provide reducing equivalents for hydrogen production.

The reversible Fe-hydrogenase is highly oxygen sensitive, thus O₂ evolution by photosynthesis must be limited in order to achieve photoproduction of H₂ by hydrogenase upon illumination of a dark-adapted culture. Establishment of anaerobiosis has been attempted by flushing the reaction vessels with inert gas (e.g. argon or nitrogen), which is expensive and impractical for scaled up cultures, and by application of exogenous reductants (e.g. sodium dithionite or herbicides to poison photosynthetic O₂ evolution), which are potentially toxic to the cells.

In 2000 at Berkeley University, in an effort to circumvent the severe oxygen sensitivity of the reversible hydrogenase by separating photosynthetic oxygen evolution (with carbon accumulation) from the consumption of cellular metabolites, a team of researchers found that sulfur deprivation of the algae Chlamydomonas reinhardtii inactivated oxygen production at PS-II (Photosystem II) in the light and caused a sharp decline in RuBisco enzyme, resulting in decreased carbon dioxide assimilation through the Calvin Benson cycle and anaerobic conditions (Melis et al., Plant Physiol 2000;122:127-135). After 2 days with full illumination, when the algae's environment had turned anaerobic and all oxygen had been consumed, the algae started producing hydrogen for few days. However, hydrogen productivity by the algae in such a system does not approach commercial viability, and the ensuing nutritional stress is damaging, and eventually toxic to the cells. Thus, in the Melis process the actual rate of hydrogen gas accumulation is at best 15 to 20% of the photosynthetic capacity of the cells [Melis and Happe 2001, Plant Physiol. November; 127(3):740-8] and hydrogen production by sulfur deprivation of the algae cannot be continued indefinitely. The yield begins to level off and decline after about 40-70 hours of sulfur deprivation, and after about 100 hours of sulfur deprivation the algae need to revert to a phase of normal photosynthesis to replenish endogenous substrates. Initiation of photoproduction of hydrogen gas (“stage 2”) lags significantly, typically 24-30 hours, until establishment of anaerobic conditions. As the potential of the algae to produce large amounts of hydrogen, using its Fe-hydrogenase enzyme, is high, efforts to enhance hydrogen productivity of this and other algae have continued, for example, by repeating cycles of light restriction and oxygen depletion with cycles of undeprived photosynthesis (see, for example, US20010053543 to Melis et al), control of photosynthesis by restriction of light energy of illumination and selection and/or genetic engineering to produce algae having limited light harvesting mechanisms (see, for example, US20080120749 to Melis), diminished sulfur uptake (see US20050014239, to Melis et al) or reduced oxygen sensitivity of their hydrogenases (see, for example, US20090263846 and US20060228774, both to King et al). Still, to date little significant progress has been made.

It has been proposed (Terauchi et al, JBC 2009; 284: 25867-878) that the main reason for low hydrogen production in this system is that during sulfur deprivation, most of the oxygen-sensitive reduced ferredoxins (PetF, FDX2) that transport electrons from PS-I to the hydrogenase are oxidized, and since the culture medium lacks sulfur, the algae cannot effectively renew levels of ferredoxin, an iron-sulfur protein. In addition, sulfur deprivation leads to decreased transcription of many components of the photosynthetic complexes, as well as enzymes and other biologically active molecules. This compounds the disadvantage of the lag period of the Melis process, typically 24-48 hours, until establishment of anaerobic conditions. Thus, by the time the environment becomes anaerobic the alga is left weakened and with diminished ferredoxin, further diminishing the photoproduction of hydrogen gas.

Co-culture of algae and photosynthetic anaerobic hydrogen producing bacteria co-culture has been proposed for algal photoproduction of hydrogen, as described above.

Algae and bacteria co-exist in nature, and efforts have been made to identify bacterial symbionts for enhanced efficiency of algal growth, for example, in bioremediation of pollutants or biomass production (see, for example, Gonzalez-Bashan et al, Can. J. Microbiol., 2000; 46:653-59; and US20100311156, to Belaiev et al).

Kawaguchi et al (J. Bioscience and Bioengineering 2001;91:277-282) have proposed growing the photosynthetic bacterium R. marinum along with Lactobacillus amylovorus and algal biomass, to metabolize the algal starches into lactate as an electron donor for bacterial hydrogen production.

U.S. Patent applications 20030162273 and 20050014239 to Melis disclose co-culturing photosynthetic, hydrogen producing algae (wild type and genetically engineered for reduced sulfate utilization) with a hydrogen-producing bacteria in order to enhance hydrogen production. However, as sulfur is a crucial component for the production of ferredoxin, with less ferredoxin in the sulfur deprived or deficient algae, electron transport to Fe-hydrogenase is diminished, and subsequently hydrogen production by the algae is low. Addition of anaerobic hydrogen-producing bacteria to the culture is intended to compensate for the loss of hydrogen productivity of the algae, caused by the reduced intake of sulfate by the algae. Further hydrogen producing capacity is achieved by the addition of an anaerobic fermentive bacteria, such as Clostridium. However, algal hydrogen production remains depressed, until traverse of the lengthy latency period and establishment of microoxic and/or anaerobic culture conditions.

### SUMMARY OF THE INVENTION

According to an aspect of some embodiments of the present invention there is provided a method of generating hydrogen gas, the method comprising sequentially

(a) propagating photosynthetic algae in a propagation medium, the propagation medium comprising sulfur;

(b) co-culturing the algae with bacteria in a culturing medium for a length of time sufficient to ensure reduced oxygen culturing conditions, wherein the culturing medium comprises a reduced amount of sulfur compared to the propagation medium;

(c) depleting at least some of the bacteria in the culturing medium to generate a bacteria-reduced culturing medium;

(d) culturing the algae in the culturing medium for a length of time sufficient to ensure anaerobic culturing conditions;

(e) culturing the algae in the culturing medium under anaerobic culturing conditions, thereby generating the hydrogen gas; and

(f) collecting the hydrogen gas.

According to an aspect of some embodiments of the present invention there is provided a method of generating hydrogen gas, the method comprising

(a) propagating photosynthetic algae in a propagation medium, the propagation medium comprising sulfur;

(b) culturing the algae in a culturing medium which comprises a reduced amount of sulfur compared to the propagation medium, for a length of time sufficient to establish anaerobic culturing conditions, wherein the culturing is co-culture with added bacteria for at least a portion of the length of time;

(c) culturing the algae in the culturing medium under anaerobic culturing conditions, thereby generating the hydrogen gas; and

(d) collecting the hydrogen gas,

wherein the length of time to anaerobic culture conditions of step (b) is reduced compared to the length of time of a similar culture of algae not co-cultured with added bacteria.

According to an aspect of some embodiments of the present invention there is to provided a system for generating hydrogen gas, the system comprising sequentially:

(a) a sealed culture vessel comprising photosynthetic algae and bacteria co-cultured in a culturing medium comprising a reduced amount of sulfur as compared to an algal propagation medium;

(b) a source of illumination of the culture vessel; and

(c) a means for collecting hydrogen gas from the culture vessel,

wherein the bacteria are comprised in a bacterial containment in fluid association with an algae containment, the algae containment separated therefrom by a fluid- and gas-permeable and bacterial impermeable barrier.

According to some embodiments of the invention, the bacteria comprises oxygen-consuming bacteria.

According to some embodiments of the invention, the method further comprising depleting at least some of the bacteria in the culturing medium to generate a bacteria-reduced culturing medium following step (b) and prior to, or during step (c).

According to some embodiments of the invention, the depleting is effected wherein oxygen consumption of the algal culture is equal to or greater than photosynthetic oxygen production of the algal culture, as measured under high intensity illumination.

According to some embodiments of the invention, the bacteria-reduced culturing medium is essentially devoid of the bacteria.

According to some embodiments of the invention, the propagation medium is essentially devoid of the bacteria.

According to some embodiments of the invention, the the culturing medium is essentially devoid of sulfur.

According to some embodiments of the invention, the culturing the algae under anaerobic conditions is effected under illuminated conditions.

According to some embodiments of the invention, the method further comprising effecting any of the steps prior to culturing the algae under anaerobic conditions under illuminated conditions.

According to some embodiments of the invention, the illumination during the step of culturing the algae under anaerobic conditions is of greater intensity than during any of the steps prior to the culturing the algae under anaerobic conditions.

According to some embodiments of the invention, all of the steps are effected under illuminated conditions.

According to some embodiments of the invention, the bacteria are comprised in a bacterial containment in fluid association with an algae containment, the algae containment separated from the bacterial containment by a fluid- and gas-permeable and bacterial impermeable barrier.

According to some embodiments of the invention, the bacterial containment is located within the algae containment and separated therefrom by the fluid- and gas-permeable and bacterial impermeable barrier.

According to some embodiments of the invention, the bacterial containment is a dialysis bag.

According to some embodiments of the invention, the bacterial containment is remote from the algae containment and in fluid association therewith via fluid connecting means and separated therefrom by a fluid- and gas-permeable and bacterial impermeable barrier.

According to some embodiments of the invention, the bacterial containment further comprises a carbon source.

According to some embodiments of the invention, the volume of the algae in the co-culture is about 5-50 times greater than a volume of the bacteria.

According to some embodiments of the invention, the volume of the algae in step (b) is about 20 times greater than a volume of the bacteria.

According to some embodiments of the invention, the co-culture comprises about 10³ to about 10⁸ algae cells per ml.

According to some embodiments of the invention, the co-culture comprises about 10⁴ to about 10⁷ algae cells per ml.

According to some embodiments of the invention, the co-culture comprises about 10⁵ to 10⁶ algae cells per ml.

According to some embodiments of the invention, the co-culture comprises about 3-6×10⁶ or about 3-6×10⁷ algae cells per ml.

According to some embodiments of the invention, the culturing in (b) and (c) is to for about 4 to about 60 hours.

According to some embodiments of the invention, the culturing in (b) and (c) is for about 10 to about 40 hours.

According to some embodiments of the invention, the the culturing in (b) and (c) is for about 30 hours.

According to some embodiments of the invention, the hydrogen gas generation is detectable after culturing the algae in (b) and (c) for about 30 hours.

According to some embodiments of the invention, the algae comprises green algae.

According to some embodiments of the invention, the algae comprises unicellular, photosynthetic algae.

According to some embodiments of the invention, the algae comprise algae having a Fe-hydrogenase enzyme.

According to some embodiments of the invention, the algae is selected from the group consisting of Platymonas subcordiformis, Rhodobacter sphaeroide and Chlamydomonas reinhardtii.

According to some embodiments of the invention, the bacteria comprises oxygen-consuming bacteria.

According to some embodiments of the invention, the bacteria comprises an obligatory aerobic bacteria.

According to some embodiments of the invention, the bacterium is Pseudomonas fluorescens.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying figures. With specific reference now to the drawings in detail, it is stressed that the particulars shown are by way of example and for purposes of illustrative discussion of embodiments of the invention. In this regard, the description taken with the figures makes apparent to those skilled in the art how embodiments of the invention may be practiced.

In the drawings:

FIG. 1 is a histogram illustrating enhanced initial hydrogen gas photoproduction from algal culture when co-cultured with bacteria. Chlamydomonas rheinhardtii was cultured in sulfur-free TAP medium in a sealed Roux bottle with mixing, along with Pseudomonas fluorescens, contained in a dialysis bag. Bacteria were removed 7.5 hours after sealing, cultures resealed, and after achieving microoxic/anaerobic conditions, gaseous evolution was detected, gas collected, analyzed and quantitated for the first 14 hours following observation of gas evolution. Hydrogen production is expressed as ml volume per liter culture. Left column-algal-bacterial co-culture. Right column-Hydrogen production in algal culture without added bacteria;

FIG. 2 is a histogram illustrating enhanced total hydrogen gas photoproduction from algal culture when co-cultured with bacteria. Chlamydomonas rheinhardtii was cultured in sulfur-free TAP medium in a sealed Roux bottle with mixing, along with Pseudomonas fluorescens, contained in a dialysis bag. Bacteria were removed 7.5 hours after sealing, cultures resealed, and after achieving microxic/anaerobic conditions, gaseous evolution was detected, gas collected, analyzed and quantitated until cessation of gas evolution following gas evolution. Hydrogen production is expressed as ml volume per liter culture. Left column-algal-bacterial co-culture. Right column-Hydrogen production in algal culture without added bacteria;

FIG. 3 is a graphic presentation of rapid and enhanced evolution of gas in algal cultures co-cultured with bacteria, compared to gas production in identical algal cultures without added bacteria. Chlamydomonas rheinhardtii was cultured in sulfur-free TAP medium in a sealed Roux bottle with mixing, along with Pseudomonas fluorescens, contained in a dialysis bag. Bacteria were removed 7.5 hours after sealing, cultures resealed, and after achieving microoxic/anaerobic conditions, gaseous evolution was detected, gas collected in a graduated cylinder by water displacement was analyzed and quantitated from time of sealing, for 72 hours, with frequent determinations during the first 12 hours. Gas production is expressed as ml volume (Y-axis) over time (hours, X-axis). Shaded diamonds (♦) algal-bacterial co-culture. Open diamonds (⋄) gas production in algal culture without added bacteria. Note the rapid kinetics of gas evolution in the algal-bacterial co-culture during the first 36 hours, and the absence of significant gas evolution in the algae-only culture.

### DESCRIPTION OF SPECIFIC EMBODIMENTS OF THE INVENTION

The present invention, in some embodiments thereof, relates to a photocatalytic method of generating hydrogen gas in algae, and, more particularly, but not exclusively, to algal-bacterial co-culture for enhancing the kinetics and improving the yield of algal hydrogen photoproduction.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Molecular hydrogen is a candidate for replacing or supplementing fossil fuels as a source of clean energy. Natural biological production of hydrogen is based on the presence of hydrogenase enzymes present in certain green algae and photosynthetic bacteria which are capable of accepting electrons from photosystem I (PSI) and conversion thereof into hydrogen gas. However, the extreme sensitivity of the FE-hydrogenase enzymes to oxygen requires anaerobic conditions for hydrogen photoproduction by this pathway. The yield of molecular hydrogen from algae using this pathway is limited for a number of reasons, one of which being the severe consequences, for the organism, of the prolonged sulfur deprivation required to initiate microoxic/anaerobic conditions while illuminated.

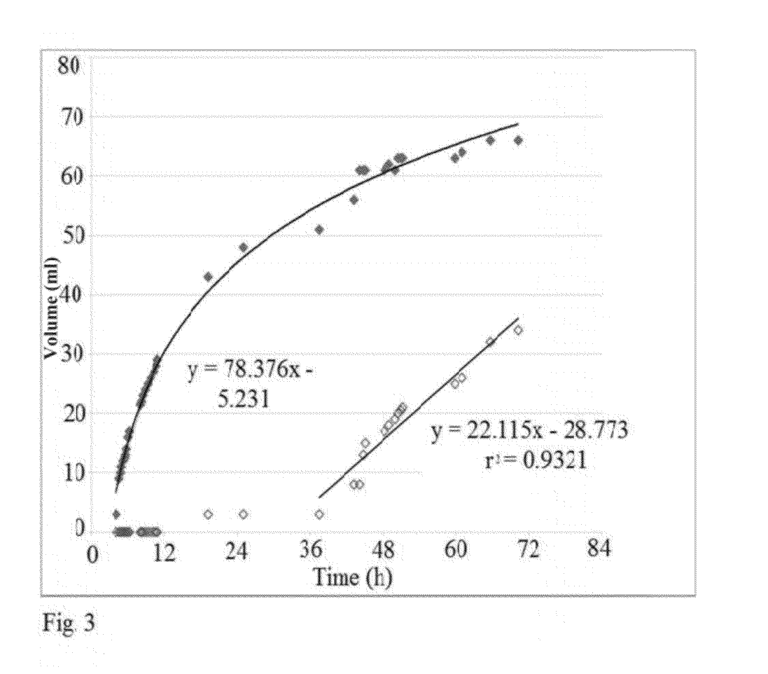

The present inventor has attempted to address this problem by adding bacteria to the algal culture during the early stages of sulfur deprivation. The present inventor has uncovered that, despite the potential toxicity of bacterial co-culture, addition of bacterial culture to a culture of photosynthetic algae, during the period of sulfur deprivation, shortens significantly the normally lengthy period of latency proceeding establishment of anaerobic culturing conditions, which, in turn, allows for more rapid hydrogen gas photoproduction by the cultured algae, as compared with a similar culture of algae cultured without added bacteria (see Example I, FIGS. I and 3). Algal hydrogen photoproduction following co-culture with bacteria was also of greater intensity than that recorded in cultures lacking added bacteria (see Example I and FIG. 3).

Thus, according to one aspect of one embodiment of the invention there is provided a method of generating hydrogen gas, the method comprising sequentially:

(a) propagating photosynthetic algae in a propagation medium, the propagation medium comprising sulfur;

(b) co-culturing the algae with bacteria in a culturing medium for a length of time sufficient to ensure reduced oxygen culturing conditions, wherein the culturing medium comprises a reduced amount of sulfur compared to the propagation medium;

(c) depleting at least some of the bacteria in the culturing medium to generate a bacteria-reduced culturing medium;

(d) culturing the algae in the culturing medium for a length of time sufficient to ensure anaerobic culturing conditions;

(e) culturing the algae in the culturing medium under the anaerobic culturing conditions, thereby generating the hydrogen gas; and

(f) collecting the hydrogen gas.

As used herein, the terms algae, alga or the like, refer to plants belonging to the subphylum Algae of the phylum Thallophyta. The algae are unicellular, photosynthetic, algae and are non-parasitic plants without roots, stems or leaves; they contain chlorophyll and have a great variety in size, from microscopic to large seaweeds. In some embodiments of the invention, green algae, belonging to Eukaryota-Viridiplantae-Chlorophyta-Chlorophyceae are used. Non-limiting examples of members of the Chlorophycae include the Dunaliellales, Volvocales, Chlorococcales, Oedogoniales, Sphaeropleales, Chaetophorales, Microsporales and the Tetrasporales. In some specific embodiments, the algae is selected from the group consisting of Platymonas subcordiformis, Rhodobacter spheroide and Chlamydomonas rheinhardtii.

In some specific embodiments, C. reinhardtii, belonging to Volvocales-Chlamydomonadaceae, is used. In other specific embodiments, the strain to Chlamydomonas reinhardtii CC125 is used. However, algae useful in the invention may also be blue-green, red, or brown, so long as the algae are able to produce hydrogen. Such hydrogen-photoproducing capability is conferred, in nature, by the presence of an Fe-hydrogenase capable of transferring electrons to hydrogen to produce molecular hydrogen gas. Thus, in one specific embodiment, the algae comprise algae having an Fe-hydrogenase. Algae suitable for use in the present invention include, but are not limited to, naturally occurring algae (wild type), cultivated strains of algae, strains of algae resulting from hybridization and selection processes and genetically modified algae, having specifically enhanced traits. For example, Melis et al. has disclosed mutant algae having reduced sulfur uptake (US20050014239), and Yacobi et al has disclosed algae having genetically modified ferredoxins and hydrogenase (see, for example, US20100203609, US20090263846). Algae having specific characteristics may also be used in some embodiments of some aspects of the invention, for example, mutant algae having modified photosensitivity or components of photosynthesis (see, for example, Grossman et al, Photosynth Res 2010;106:3-17). Mutant algae and methods for their production and screening are disclosed by, inter alia, Plummer et al (US20100273149) and Hankamer et al (US20090221052).

According to some embodiments, the algae are provided as isolated, purified algal cultures. In other embodiments, the algal propagation cultures are essentially devoid of the bacteria comprised in the bacterial containment.

General methods for culture of Chlamydomonas are well known in the art, and are described in detail in The Chlamydomonas Handbook (Harris, San Diego Calif., Academic Press, 2009, the contents of which are incorporated herewith by reference). General methods for photoproduction of hydrogen in algae are described in detail in Hemschemeir et al (Photosynth Res 2009;102:523-40), the contents of which are incorporated herewith by reference.

As used herein, the word “culture” refers to the maintenance of living cells in media that is conducive to their ongoing viability. Many media are conducive to not only viability, but also growth under the appropriate environmental conditions. As used herein, the term “growth” is defined as expansion of the culture, i.e. increase of number of organisms in the culture, over a defined period of time. The most common growth media include broths, gelatin, and agar, all of which will include sulfur as a component. The culture may be solid or liquid. Culturing may be done on a commercial scale, or in a single Petri dish.

As used herein, the term “propagation medium” refers to a medium conducive to growth of the algae under appropriate environmental conditions. Propagation media, as used herein, typically comprise sulfur compounds, in amounts sufficient to maintain photosynthesis in photosynthetic algae. One non-limiting example of a propagation medium suitable for use in some embodiments of the present invention is TAP, Tris-acetate-phosphate, including sulfur compounds. In some embodiments, the propagation medium comprises from about 0.05 to about 0.25 millimolar sulfur, as MgSO₄, FeSO₄, ZnSO₄ and/or CuSO₄. In a specific embodiment, the propagation medium comprises about 0.1 to about 0.15 millimolar sulfur. According to some embodiments of the present invention, the propagation medium is devoid of the bacteria comprised in the bacterial containment.

As used herein, the term “culturing medium” refers to a medium for maintaining the algae in a viable state, with little or no growth, for the duration of the culture period. In one embodiment, the culturing medium has a reduced amount of sulfur, as compared to the propagation medium, so that culture of the photosynthetic algae in the reduced-sulfur culture medium results in inhibition of oxygenic function of the photosynthetic pathways, leading to microoxic or, ostensibly anaerobic conditions. Culturing media suitable for use with the present invention include, but are not limited to, TAP medium in which the sulfur compounds (e.g. sulfates) have been replaced by equimolar equivalents of chloride containing compounds. Aerobic state can be monitored in the algal containment by measurement of dissolved oxygen in the culture medium or in samples of the culture medium. Dissolved oxygen can be measured, for example, using a Clark electrode.

In some embodiments, the sulfur content (molar equivalents/liter) of the culture medium is about 50%, about 40%, about 20%, about 10%, about 0.08%, about 0.05%, about 0.01% or less of the sulfur content (molar equivalents/liter) of the propagation medium. In another, specific embodiment the culture medium is essentially devoid of sulfur compounds.

It will be appreciated that transfer of the algae from propagation medium to sulfur-poor culture medium entails washing of the algae, in order to remove traces of sulfur. Algae can be washed by harvesting by mild centrifugation (for example, 2-3 minutes at 3,500-5000 g at room temperature), gentle resuspension in the desired medium. This may be repeated as necessary to remove sulfur compounds.

As used herein, the term “co-culture” refers to simultaneous culture of two or more organisms within the same culture system. One non-limiting example of algal co-culture is the simple addition of a second organism (e.g. bacteria) to an algal culture, under conditions sufficient for the maintenance of viability of both the algae and the additional organism, and/or growth of one organism or the other or both. It will be appreciated that the “co-culture”, as used herein, refers to a man-made culture which does not exist in nature at least in terms of the bacterial/algae type or the components and/or their concentration.

In some embodiments of the present invention, algae are co-cultured with bacteria, in order to shorten the latency period between sulfur deprivation and establishment of microoxic and/or anaerobic conditions for hydrogen generation by the algae. In one specific embodiment, the bacteria are oxygen-consuming bacteria, such as obligate aerobic or facultative anaerobic bacteria. Microaerophilic bacteria, anaerobic bacteria and aerotolerant bacteria do not consume significant amounts of oxygen, but can be suitable for use with the present invention if found to contribute to reduction of dissolved oxygen when co-cultured with photosynthetic algae in reduced sulfur culture medium. A non-limiting list of oxygen-consuming bacteria suitable for use with the present invention includes the Bacillus, Nocardia, Mycobacterium, Pseudomonas and the like. In a particular embodiment the aerobic bacteria is a Pseudomonas bacteria. In some specific embodiments, the aerobic bacterium is Pseudomonas fluorescens, and the algae is Chlamydomonas reinhardtii.

The algal containment can comprise algae cultured at a number of cell densities. The algal density in culture or in co-culture can comprise about 10³ to about 10⁸ algae cells per ml, about 10⁴ to about 10⁷ algae cells per ml, about 10⁵ to about 10⁶ algae cells per ml. In one specific embodiment, the algal density in the co-culture comprises 3-6×10⁶ cells per ml. In another specific embodiment, the algal density in the co-culture comprises 3-6×10⁷ cells per ml. Bacterial cells are typically used from fresh, mid-log-phase bacterial cultures, which can be diluted up to 1:10 or more before establishment of to the microoxic/anaerobic conditions. In a specific embodiment, for each liter of co-culture, mid-log phase bacterial cells from 1 liter bacterial culture are pelleted, diluted approximately 1:10 in culture medium, and a volume of the diluted bacterial culture introduced into the bacterial containment according to the ratios detailed herein.

The algal-bacterial co-culture can comprise varying ratios of algae to bacterial microorganisms, from about a 1:1 algae to bacteria ratio, to about 1:2, about 1:3, about 1:4, about 1:5, about 1:10, about 1:20, about 1:30, about 1:40, about 1:50, about 1:60, about 1:70, about 1:80, about 1:90, about 1:100, about 1:150, about 1:200, about 1: 400, about 1:500, 1:1000 algae to bacteria, or more. The ratio of algae to bacteria in algal-bacterial co-culture can also be expressed in terms of volume thus, according to some embodiments of the present invention the algal and bacterial components of the co-culture are separated, thus the co-culture comprises an algae containment and a bacterial containment, and the volume of the algae culture in the co-culture is about 1-100 times greater than the volume of the bacteria in the bacterial containment, about 5-50 times greater than the volume of the bacteria in the bacterial containment, about 10-40 times greater than the volume of the bacteria in the bacterial containment, about 20-30 times greater than the volume of the bacteria in the bacterial containment and about 20-25 times greater than the volume of the bacteria in the bacterial containment. In a specific embodiment, the volume of algal culture in the co-culture is about 20 times greater than the volume of the bacteria in the bacterial containment, e.g. about 50 ml bacterial culture in the bacterial containment to about 1 liter of algal culture in the algal containment.

According to some embodiments, the bacteria and algae are co-cultured in separate containments. In some embodiments, the separation of containments is in order to improve illumination efficiency of the algal culture. In other embodiments, separation is to allow simple introduction and removal of the bacterial containment into the system, for example, reduction and/or removal of the bacterial culture after approaching microoxic/anaerobic conditions following sulfur starvation, or reduction or removal of the bacterial culture before collecting hydrogen gas from the algal culture.

According to some aspects of the present invention, the algal and bacterial containments are in fluid association and separated from one another by a fluid permeable and gas-permeable, but bacterial impermeable barrier. As used herein, the term “fluid association” refers to the ability of fluids to move between the algal and to bacterial containments. Such fluid association can be direct fluid association, in which, for example, the bacterial containment is immersed within the medium of the algal containment, or remote and in indirect fluid association, e.g. by means of fluid connectors such as pipes, tubing, channels, conduits, and the like. In one embodiment, a remote, indirect fluid association comprises a vessel for the algal containment and a separate, remote vessel for the bacterial containment, connected by suitable tubing (e.g. plastic, glass, rubber, stainless steel), optionally further comprising pumping means, filtering means and control means (e.g. valves) for circulating the medium between and through the two containments. The algal and bacterial containments may be in flasks, tanks, pools, sleeves, counter-current devices, hollow fibers and the like, or in specially designed bioreactors. The algal and bacterial containments can be of any dimensions, for example, and can contain volumes in a range from about 0.1 to 1 liter, 1 liter to about 10 liters, 10 liter to about 1000 liters, 1000 about 10,000 liters, 50,000 liters or more. In the case of large pools or bioreactors, volumes of 10 s to 100 s, 1000 s, and more cubic meters are contemplated. In one specific embodiment, the algal containment is a 1.1 liter Roux bottle and the bacterial containment is a 50 ml dialysis bag. Methods and bioreactors for co-culture of algae and bacteria are well known in the art, and described in detail in, for example, The Chlamydomonas Handbook (Harris, San Diego Calif., Academic Press, 2009, the contents of which are incorporated herewith by reference). Special photobioreactors and methods for their use are described in detail by Eriksen (Biotechnol Letters, 2008;1525-36, the contents of which are incorporated herewith fully by reference). In some embodiments, the bacterial containment further comprises a source of carbon, for example glucose, starch, lipids, proteins, etc.

The production of hydrogen is carried out under “microoxic conditions” which refers conditions in which a minimal oxygen concentration is maintained so as to avoid hydrogenase inactivation, and generally refers to a substantially anaerobic environment. In order to establish microoxic/anaerobic conditions, the algal or algal-bacterial culture is sealed following introduction of sulfur-poor culturing medium. Sealing can be via any means of excluding exposure to air or ambient gases, such as flexible rubber or neoprene seals, glass, plastic or rubber stoppers, wax, etc, or via two- or three- or more-way valves which can be set to exclude gases. Optionally, the culture can be flushed with an inert gas (e.g. argon) following introduction of sulfur-poor culturing medium.

According to one aspect of some embodiments of the invention, the algal and bacterial containments are separated one from the other by a gas- and fluid-permeable, but bacterial impermeable barrier. Such a barrier typically comprises a porous filter, and/or membrane having pores small enough to exclude the bacterial cells, but large enough to allow free passage of fluid and small molecule components of the medium. Such a barrier may comprise a Micropore™ or Millipore™ filter, with permeability of less than 50 nm pore size, situated in a suitable filter housing interposed in the fluid connectors between the algal and bacterial containments. In another embodiment, the barrier is an integral part of the septum or wall between the algal and bacterial containments.

In one specific embodiment of the present invention, the bacterial containment and algal containment are in direct fluid association, the bacterial containment being immersed within the medium of the algal containment. In such an embodiment, the barrier can comprise a large portion, or even the entire surface of the bacterial containment. In one non-limiting example, the bacterial containment comprises a bag or sleeve fashioned from the gas- and fluid-permeable, but bacterial impermeable barrier, such as a dialysis bag. In one specific embodiment, the bacterial containment is a sealed dialysis bag, and the barrier is the cellulose or cellulose-like dialysis membrane permeable to molecules up to 6 kD, thus allowing free circulation of the fluid and small molecular (e.g. salts and organic solutes) components between the bacterial and algal containments, but excluding live and dead algal and bacterial cells, as well as macromolecular debris. Such a bacterial containment allows for ease of introduction and removal from the medium of the algal containment.

Thus, according to some embodiments of the present invention, following co-culturing with the algae, the bacterial bacteria in the culture medium is depleted to generate a bacteria-reduced culturing medium. Depletion of the bacteria and generating the bacteria-reduced medium can be effected by removing a portion (e.g. 1%, 5%, 10%, 20%, 40%, 50%, 75% or more) of the bacteria from the bacterial containment, effectively reducing the number of bacteria in the culture medium. In some embodiments, about 100% of the bacteria are depleted, producing an essentially bacteria-free culture medium. Where the bacterial and algal containments are in indirect fluid association, this can be easily accomplished by interrupting the fluid connection to between the containments, as with a valve or stopper. Where the fluid association is direct, as where the bacterial containment is immersed within the algal containment, the bacterial containment can be removed from the algal containment by simple mechanical removal (e.g. removal of a dialysis bag), resulting in an essentially bacteria-free culture medium in the algal containment. According to some embodiments, the duration of algal and bacterial co-culture, until depletion of the bacteria, is about 0.5, about 1, about 1.5, about 2, about 2.5, about 3, about 3.5, about 4, about 4.5, about 5, about 5.5, about 6, about 6.5, about 7, about 7.5, about 8, about 8.5, about 9, about 10, about 11, about 12, about 13, about 15, about 17, about 20, about 25, about 30, about 35, about 40 hours or more. In some embodiments, the duration of algal and bacterial co-culture is about 30 hours. In other embodiments, the duration of algal and bacterial co-culture is about 15 hours. In other embodiments, the duration of algal and bacterial co-culture is about 7.5 hours. In other embodiments, the duration of algal and bacterial co-culture is about 4 hours.

In some embodiments, the duration of algal and bacterial co-culture is until equilibrium is reached between photosynthetic oxygen evolution and cellular respiratory oxygen consumption, e.g. algal oxygen consumption by cellular respiration is equal to or greater than algal oxygen evolution by photosynthesis, under high intensity illumination. The oxygen consumption of the algal culture is measured by measuring dissolved oxygen, over a predetermined period of time (e.g. 5 minutes) in a sample of the culture, or the entire culture, for example, using a Clark electrode (with and without sodium bicarbonate), or gas chromatograph, while the culture or sample is without illumination sufficient for photosynthesis. Oxygen evolution or production by photosynthesis is measured by measuring dissolved oxygen, over a predetermined period of time (e.g. 5 minutes) in a sample of the culture, or the entire culture, for example, using a Clark electrode, or gas chromatograph, while the culture or sample is brightly illuminated, at least sufficiently bright for photosynthesis. In some embodiments, algal and bacterial co-culture is begun immediately following transfer of the algal culture to sulfur depleted culture medium, and the bacteria are partially, or completely depleted from the co-culture at the point at which the measurements indicate that oxygen consumption by the algal culture is equal to, or greater than, the photosynthetic oxygen production by the algae, as measured under high intensity illumination. In some embodiments, to equilibrium is reached at about 1, about 1.5, about 2, about 2.5, about 3, about 3.5, about 4, about 4.5, about 5, about 5.5, about 6, about 6.5, about 7, about 7.5, about 8, about 8.5, about 9, about 10, about 11, about 12, about 13, about 15, about 17, about 20, about 25, about 30, about 35, about 40, about 45, about 50, about 55 or more hours. In some embodiments, the equilibrium between photosynthetic oxygen evolution and cellular respiratory oxygen consumption is reached in about 30 hours. In other embodiments, the equilibrium between photosynthetic oxygen evolution and cellular respiratory oxygen consumption is reached in about 15 hours. In other embodiments, the equilibrium between photosynthetic oxygen evolution and cellular respiratory oxygen consumption is reached in about 7.5 hours. In other embodiments, the equilibrium between photosynthetic oxygen evolution and cellular respiratory oxygen consumption is reached in about 4 hours.

According to another aspect of some embodiments of the present invention, following depletion of the bacteria from the culture medium, the algal containment is sealed, and the algae are cultured in the culture medium for a length of time sufficient to ensure microoxic/anaerobic conditions, critical to the photoproduction of hydrogen gas in the photosynthetic algae. According to some embodiments, the duration of culturing, from commencement of the algal-bacterial co-culture, until establishment of microoxic/anaerobic conditions is about 1, about 1.5, about 2, about 2.5, about 3, about 3.5, about 4, about 4.5, about 5, about 5.5, about 6, about 6.5, about 7, about 7.5, about 8, about 8.5, about 9, about 10, about 11, about 12, about 13, about 15, about 17, about 20, about 25, about 30, about 35, about 40, about 45, about 50, about 55, about 60 hours or more. In some embodiments, culturing, from commencement of the algal-bacterial co-culture, until establishment of microoxic/anaerobic conditions is for about 4 to about 60 hours, about 10 to about 40 hours, and about 30 hours.

According to another aspect of some embodiments of the present invention, following establishment of microoxic/anaerobic conditions in the sealed algal culture, the algal are further cultured under the microoxic/anaerobic conditions to generate hydrogen gas. According to some embodiments, the duration of culturing under microoxic/anaerobic conditions is about 1, about 1.5, about 2, about 2.5, about 3, about 3.5, about 4, about 4.5, about 5, about 5.5, about 6, about 6.5, about 7, about 7.5, about 8, about 8.5, about 9, about 10, about 11, about 12, about 13, about 15, about 17, about to 20, about 25, about 30, about 35, about 40, about 45, about 50, about 55, about 60, about 75, about 80, about 90, about 100, about 110, about 120 hours, about 6, about 7, about 8, about 9 about 10 or more days. In some embodiments the duration of culturing under microoxic/anaerobic conditions is until hydrogen gas evolution is no longer detected.

Algal cultures can be reused following cessation or significant loss of efficiency of hydrogen photoproduction. The algae can be “rejuvenated” by return to propagation medium (following sufficient washing and resuspension), under aerobic conditions and illumination for a period of time sufficient for the algae to re-establish vigorous photosynthesis and growth, followed by another cycle of culture for hydrogen photoproduction according to methods of the present invention. Reuse of algal culture can be further facilitated by affixing the algae to a three dimensional, semi-solid or solid support, matrix or gel member, such as alginate or plastic beads, fibers, mats, sheets, etc., which can be easily removed from the culture or propagation medium, washed and transferred to a fresh medium.

Collection, detection, purification and calculations of yield of hydrogen gas from algal culture are well known in the art, and described in detail in, for example, The Chlamydomonas Handbook (Harris, San Diego Calif., Academic Press, 2009, the contents of which are incorporated herewith by reference) and Hemschemeir et al (Photosynth Res 2009;102:523-40), the contents of which are incorporated herewith by reference. According to one specific embodiment, the evolved gas from the algal culture is expelled via a tube, collected by water displacement, volume recorded and gaseous components assayed by, for example, Clark electrodes, and/or chromatographic devices, such as a gas chromatograph.

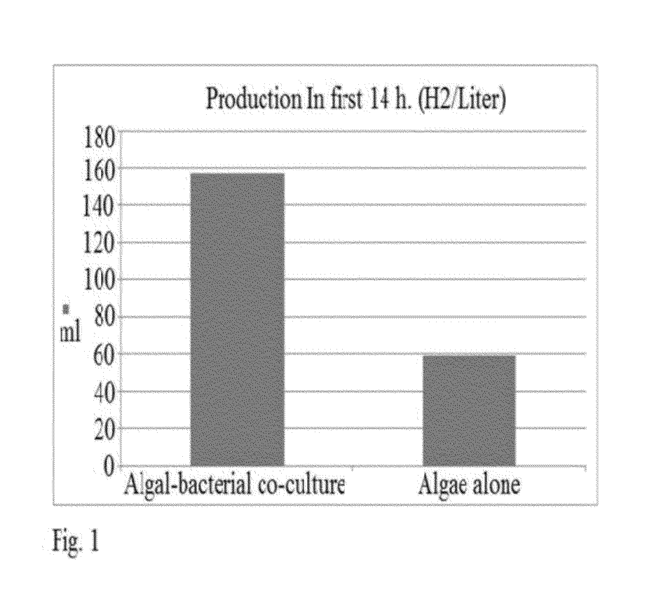

As shown herein, algal cultures co-cultured with added bacteria according to the methods of the present invention generate hydrogen gas more rapidly and in greater amounts than similar cultures without added bacteria (see Example I, and FIGS. 1, 2 and 3 herein). Shortening the length of time from commencement of culturing the algae in reduced sulfur medium to establishment of midrooxic/anaerobic culture conditions, under which hydrogen gas can be photoproduced, is of extremely great significance, both in terms of the viability of the algae in culture, and in terms of the commercial value of algal hydrogen photoproduction, as compared to competing methods for algal production. Thus, according to some aspects of some embodiments of the present to invention, there is provided a method of generating hydrogen gas, the method comprising

(a) propagating photosynthetic algae in a propagation medium, the propagation medium comprising sulfur;

(b) culturing the algae in a culturing medium which comprises a reduced amount of sulfur compared to the propagation medium, for a length of time sufficient to establish anaerobic culturing conditions, wherein the culturing is co-culture with added bacteria for at least a portion of the length of time;

(c) culturing the algae in the culturing medium under anaerobic culturing conditions, thereby generating the hydrogen gas; and

(d) collecting the hydrogen gas,

wherein the length of time to anaerobic culture conditions of step (b) is reduced compared to the length of time of a similar culture of algae not co-cultured with added bacteria.

In some embodiments, the length of time to anaerobic conditions is reduced to about 90%, about 80%, about 75%, about 70%, about 60%, about 50%, about 40%, about 30%, about 25%, about 20%, about 15%, about 10% or less the length of time to aerobic conditions in a similar culture of algae not co-cultured with added bacteria.

In some embodiments, of the present invention, the culturing, from commencement of the algal-bacterial co-culture, until establishment of microoxic/anaerobic conditions is for about 4 to about 60 hours, about 10 to about 40 hours, and about 30 hours. In some embodiments, the length of time to microoxic/anaerobic conditions of the co-cultured algal culture is about 50% that of the length of time to microoxic/anaerobic conditions of the non-co-cultured algal culture.

Thus, in some embodiments, the at least a portion of the length of time sufficient for establishing microoxic/anaerobic conditions is about 1, about 1.5, about 2, about 2.5, about 3, about 3.5, about 4, about 4.5, about 5, about 5.5, about 6, about 6.5, about 7, about 7.5, about 8, about 8.5, about 9, about 10, about 11, about 12, about 13, about 15, about 17, about 20, about 25, about 30, about 35, about 40, about 45, about 50, about 55, about 60 hours.

Photoproduction of the hydrogen gas by algal culture requires illumination. In some embodiments, illumination is provided during culturing the algae under to microoxic/anaerobic conditions. In some embodiments, illumination is provided throughout any or all steps of the method. In other embodiments, a period of dark adaptation can be optionally included during the establishment of microoxic/anaerobic conditions. In some embodiments, the dark period extends from the beginning of sulfur deprivation (depletion) of the algae for 1, about 1.5, about 2, about 2.5, about 3, about 3.5, about 4, about 4.5, about 5, about 5.5, about 6, about 6.5, about 7, about 7.5, about 8, about 8.5, about 9, about 10 or more hours. In other embodiments, the dark period extends from sulfur deprivation for 5 hours.

In some embodiments, intensity of illumination is varied during different portions of the method. For example, during propagation of the algae illumination can be in the range of 100-250 μmol photons m⁻² sec⁻, and illumination may be of lower intensity during the sulfur deprivation and establishment of microoxic/anaerobic culture conditions, and then increased during culture under microoxic/anaerobic culture conditions for hydrogen generation and collection. Factors for consideration of determining the intensity of the illumination include, but are not limited to the photosensitivity of the algae in culture, the density of the algae in the culture and light permeability/opacity of the algal culture, metabolic consequences of culture under sulfur depletion and microoxic/anaerobic conditions (for example, generation of free radicals, metabolic waste products, etc).

Illumination is typically provided externally. Illumination can be natural illumination, such as sunlight, or artificially produced and provided. For sunlight, the methods of the present invention are typically practiced out of doors, utilizing the sunlight available during the daytime. Additional artificial illumination can be added during darkness. Reduced illumination, if desired, can be achieved by shading the vessels, bioreactors, tanks, pools, or other algal containments. In some embodiments, illumination is optionally provided internally, i.e. from within the algal containment, for example, by lighting means submerged within the algal or algal-bacterial culture medium. In another embodiment, the algal containment is designed around the light source.

Artificial illumination can be provided by incandescent, fluorescent, LED or other sources. In some embodiments, the illumination is via fluorescent or LED lighting, in order to minimize the amount of heat generated during intense illumination. During illumination for photoproduction of hydrogen, the light may be from an artificial source or natural sunlight, and must be sufficient for photosynthesis to occur. In one embodiment the light intensity is between 15 and 3100 μmol photons m⁻² sec⁻¹ (and all ranges within this range such as 100-3000, 1000-2000, 1200-1800 and so on) and illumination continues for up to 120 hours (but may be for a lesser period such as 24, 48, 64 or 96 hours). Optionally, a source of high intensity illumination providing about 1,300 μmol photons m⁻² sec⁻¹ is used. In some embodiments, illumination during photoproduction of hydrogen is 80 μE. In some embodiments, illumination during photoproduction of hydrogen is 200 μE.

As algae are marine organisms, it can be advantageous to optimize the wavelength of the illumination to those most effective in the photosynthetic pathways. In wild type, and many modified algae, actinic illumination (similar to sunlight filtering through water) is most effective, and can be achieved by illuminating through a solution of 1% w/v CuSO₄.

IN some embodiments of the present invention, the algal culture and/or co-culture with bacteria are effected at ambient temperature. In other embodiments, the temperature is controlled, for example, to maintain about 25° C. in the culture. Methods for temperature control in bioreactors are well known in the art.

Further according to some aspects of the present invention, there is provided a system for generating hydrogen gas, the system comprising:

(a) a sealed culture vessel (or vessels) comprising photosynthetic algae and bacteria co-cultured in a culturing medium comprising a reduced amount of sulfur as compared to an algal propagation medium;

(b) a source of illumination of the culture vessel; and

(c) a means for collecting hydrogen gas from the culture vessel,

wherein the bacteria are comprised in a bacterial containment in fluid association with an algae containment, the algae containment separated therefrom by a fluid- and gas-permeable and bacterial impermeable barrier.

The system of the invention can further comprise means for stirring the bacterial and/or algal cultures in their respective containments, means for temperature control of the bacterial and algal containments, means for sampling the culture medium and gas from the algal and/or bacterial containments or gas collection means, and suitable means for sealing the algal containment for establishment and maintenance of microoxic/anaerobic culture conditions. The systems of the present invention may be to connected in a plurality of systems, with suitable common fluid connection means between the algal and bacterial containments, pumping, circulating and flow regulating means, filtering means and common hydrogen gas collection means. The culture vessels are preferably fashioned from a transparent or translucent material, to allow penetration of light. Photobioreactors and methods for their use are described in detail by Eriksen (Biotechnol Letters, 2008;1525-36, the contents of which are incorporated herewith fully by reference).

Hydrogen produced and collected by the methods and systems of the present invention can be stored as compressed gas, liquefied gas, by cryopreservation, chemically as compounds that release hydrogen upon heating, and the like. Stored hydrogen can be used for ammonia production, conversion of petroleum to lighter fuels (hydrocracking), in fuel cells, and the like.

It is expected that during the life of a patent maturing from this application many relevant methods will be developed and the scope of the term photoproduction of hydrogen is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example I

### Rapid and Enhanced Photoproduction of Molecular Hydrogen in Algal-Bacterial Co-culture

In order to determine whether simultaneous culture of aerobic bacteria can hasten anaerobiosis during sulfur depletion and enhance the efficiency of hydrogen photoproduction in photosynthetic algae, bacteria were co-cultured with the green algae, and the kinetics and volume of hydrogen production was determined.

Materials and Methods

Algae propagation: The algae Chlamydomonas reinhardtii strain CC125, was grown in Tris-acetate-phosphate solid medium, pH7.0, in a Petri dish.

The alga was seeded in 6 plastic flasks of 40 ml. in 20 ml. of the Tris-acetate-phosphate (TAP) medium (pH=7.0), containing sulfur compounds, in each of them, and was incubated with shaking (80 RPM) under cool white continuous illumination intensity of 200 μmol photon·m²·sec⁻¹ and at a temperature of 25° C. for 48 hours. After reaching 0.2 of the algae's logarithmic growth phase (1.2×10⁶ cells/ml) the cultures in the flasks were mixed with 2.5 liters of TAP medium in 2—1.1 Liter—Roux bottles and grown under continuous cool white illumination intensity of 200 μmol photon·m²·sec⁻¹ with continuous stiffing and bubbling of 5% CO₂ in air until reaching late logarithmic growth phase (˜3×10⁷ cells/ml). Culture density was measured by cell counting with hemaecytometer.

Bacterial culture: The bacterium Pseudomonas fluorescens was seeded in 1.0 Liter of LB medium supplemented with 100 μgram/ml Ampicillin with shaking (200 RPM), transferred to a 2.0 liter Erlenmeyer flask and incubated at 30° C. for 4 hours until reaching mid logarithmic growth phase.

Sulfur deprivation and photoproduction of hydrogen: Algae culture was harvested in late logarithmic growth phase by centrifugation (6,000 RPM for 10 minutes) and was washed 3 times with TAP (pH 7.0) medium without sulfur compounds (sulfur compounds were replaced by equivalent molar of chloride compounds), and transferred to 2 reactors each of 1.1 liter reactors (Roux bottles) with TAP-S (sulfur-free TAP) solution (pH 7.0).

The reactors were illuminated by cool white fluorescent light illumination at 200 to μmol photon m² sec⁻¹. During the first 30 hours of sulfur deprivation, samples of the culture were removed from the reactor. Measurements of oxygen respiration rate in the samples were conducted in the dark, followed by measurements of photosynthetic oxygen production and oxygen respiration in the light.

After 30 hours, when photosynthetic oxygen production had been reduced to equal to or less than oxygen respiration, the reactor was sealed with a silicon rubber septum.

Evolved gas was collected by water displacement in a graduated cylinder.

At the end of the incubation, the gas in the head space of the reactor was sampled, and the amount of hydrogen gas produced (concentration×volume) was determined.

Bacterial co-culture: The bacterial culture (1 liter) was pelleted in a centrifuge and the supernatant liquid was separated. The culture then was washed, suspended in 50 ml. of sulfur-free TAP, pH 7.0, as used for the algae, and put in a dialysis bag (6 Kd cutoff) filled fitted to the reactor size. 0.5% Glucose was added. The bacterial culture was added to the algae culture in the beginning of sulfur deprivation.

Hydrogen gas collection and measurement: Hydrogen gas was collected from the reactor during hydrogen production by water displacement in a graduated cylinder, or was sampled from the head space of the reactors, as described above. Hydrogen was measured in 1.0 ml. samples by gas chromatography in a TCD detector (30 meters) column, at a temperature of 50° C. Nitrogen was used as a carrier gas). The volume of hydrogen in the gas mixture was calculated according to a standard of pure hydrogen.

Dissolved oxygen measurement: Dissolved oxygen was measured by a Clark-type electrode. Measurements of oxygen respiration rate in the dark followed by measurements of photosynthetic oxygen production rate, minus oxygen respiration rate in the light, with and without sodium bicarbonate, were made on 3 ml. samples of culture taken from the reactor, for 5 minutes for each measurement. The samples were illuminated by a slide projector at an intensity of 1,300 μmol photon·m²·sec⁻¹. Light was filtered by a 40 ml. plastic flask filled with a solution of 1.0% CuSO₄ (w/v).

Results

Rapid Anoxia and Photoproduction of Hydrogen Gas with Algal-Bacterial Co-Culture:

Chlamydomonas reinhardtii strain CC125 was cultured with Pseudomonas fluorescens.

In a first experiment, 1 liter of 3-6×10⁶ algae per ml were cultured with the 50 ml P. fluorescens in a 1.1 liter Roux bottle. Concentration and oxygen respiration by the bacteria and algae was measured, and light intensity of 80 micro Einstein m-2 s-1. After 7.5 hours, as the dissolved oxygen level dropped, oxygen respiration by the bacteria ceased and the dialysis tubing containing the bacteria was removed from the reactor. In the algal culture without bacteria, under 200 micro Einstein m-2 s-1 of light intensity, during the sulfur deprivation, if no sodium bicarbonate is added, the rate of photosynthetic oxygen production by the algae in the light was equal or less than oxygen respiration in the dark after 40-42 hours. When algae were co-cultured from sulfur deprivation with bacteria, microoxic/anaerobic conditions were achieved about 18 hours from the start of incubation. Once anaerobic, the cultures start producing hydrogen. (In the absence of bacteria, the time taken to reach anaerobic conditions is about 40-50 hours). Hydrogen gas production increased above the level of that in the control culture without the bacteria, and was determined to be at least 3.48 ml. per hour per liter of culture, compared to 1.62 ml per hour per liter of culture without added bacteria. In this experiment, hydrogen production of the co-cultured algal culture was sustained for 14 hours. Gas collected in the graduated cylinder (under water) was 20 ml, of which 8 ml was determined to be hydrogen, and gas in the headspace was 260 ml, of which 44 ml was hydrogen.

In a second example, anoxia and photoproduction of hydrogen were evaluated over a longer period of time. Wild type Chlamydomonas reinhardtii (strain CC125) and Pseudomonas fluorescens were used. Algae was prepared in 1.1 liter Roux bottles, as in the first experiment, and illuminated with light intensity of 200 micro mol photons/m2×s. 50 ml of bacterial culture grown to 0.35 of logarithmic growth phase at 30 degrees Celsius were pelleted, and put in a 50 ml of dialysis bag, 4 hours after initiation of sulfur deprivation and a dark period of 5 hours. The cultures were then sealed with a silicon rubber septum, and illuminated at 80 μE. Measurements of photosynthetic oxygen production and oxygen respiration were conducted during the first 30 hours. According to measurements of the sampled medium, the algae in the co-culture culture, at the density of 3-6×10⁷, reached anoxia 40 hours after sulfur deprivation, while the control took 73 hours. Illumination during photoproduction of hydrogen was at 80 μE. Hydrogen evolution was first detected at 45 hours after sulfur deprivation, while in controls it was detected at 78 hours. Total volume of algal-bacterial co-culture at the end of the experiment was 1,073 ml. Total volume of hydrogen collected was 210 ml. Hydrogen evolution continued for 70 hours in co-cultured cultures, in controls 51 hours. Average hydrogen evolution per hour per liter for co-cultured algae was 2.8, for controls 1.6. Gas collected in the graduated cylinder (under water) was 66 ml, of which 24 ml was determined to be hydrogen, and gas in the headspace was 225 ml, of which 187 ml was hydrogen. In controls, gas collected in the graduated cylinder (under water) was 34 ml, of which 8 ml was determined to be hydrogen, and gas in the headspace was 239 ml, of which 112 ml was hydrogen.

Comparison of the performance of algal cultures with and without bacterial co-culture (for the first experiment) clearly reveals the advantage of adding bacteria for algal hydrogen production. Visible hydrogen evolution (bubbles in the culture) was detected at 56 hours post sulfur deprivation. Hydrogen production rate of the algal-bacterial co-culture system (algae that was co-cultured with bacteria in the incubation period) in the first 14 hours of production was: 11.21 ml/hour per 1.0 liter of culture (156 ml hydrogen per liter total), at a cell density of 3-6×10⁷ cells/ml, while hydrogen production rate of the algae alone control cultures in the first 14 hours of production was 4.2 ml/h per 1.0 liter of culture (58.8 ml hydrogen per liter total), at a cell density of 3-6×10⁶ cells/ml (FIG. 1). Thus, hydrogen photoproduction during the first 14 hours of production was enhanced 2.7 fold by combining the aerobic bacterial culture with the photosynthetic algal culture.

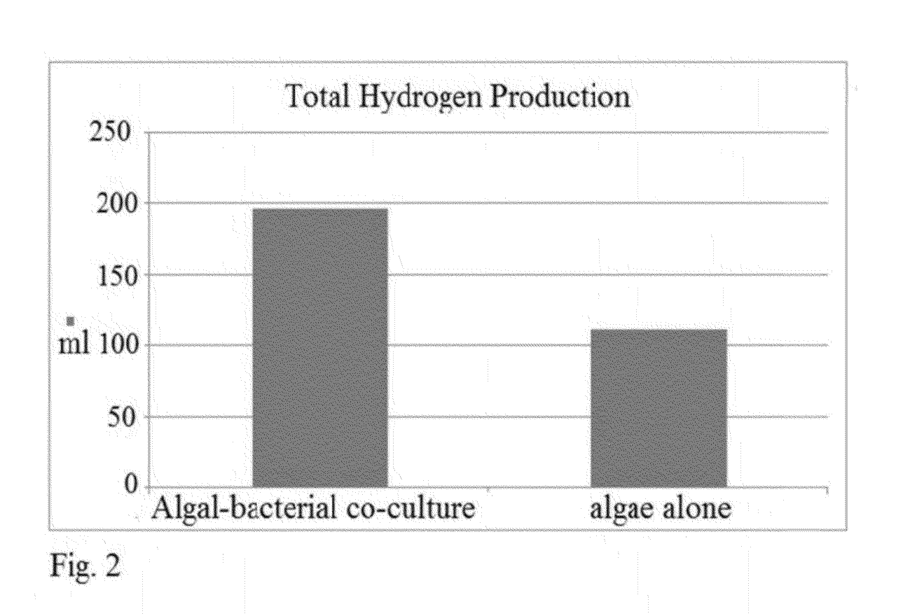

In the second experiment, total hydrogen photoproduction (combined hydrogen in the headspace and collected by water displacement), measured at 140 hours post sulfur deprivation (135.5 hours from commencement of high intensity illumination) was 196 ml for the algal-bacterial co-culture system, while the control algae alone cultures produced a total of 111 ml hydrogen per liter culture(see FIG. 2).

Measurement of gas evolution from the algal-bacterial co-culture system and from the algae only control revealed surprising differences in total gas production by the two systems. When the gas volume measured by water displacement in the graduated cylinder was monitored at frequent intervals during the incubation following sealing, significantly more rapid kinetics of gas evolution, and greater gas evolution capability were observed in the algal-bacterial co-culture system, as compared to the algae only controls. FIG. 3 shows the latency period of >36 hours before significant gas evolution in the control, algae only cultures, while gas was collected from the algal-bacterial co-cultures already before 6 hours in culture had passed (FIG. 3, shaded diamonds). Although the rapid kinetics of the first 18 hours were not sustained afterwards, FIG. 3 clearly shows the superior gas production capability of the algal bacterial co-culture system, as compared to the algae alone control under identical conditions. For example, the algal-bacterial co-culture system achieved 35 ml of gas collected at less than 24 hours, while the same volume of gas (35 ml) represented the maximal gas volume collected by water displacement in the algae-only control system (see FIG. 3).

These results indicate that, using a photosynthetic green algae such as Chlamydomonas reinhardtii, and an aerobic bacteria such as Pseudomonas fluorescens, the latency period for photoproduction of hydrogen is significantly reduced, and the intensity of hydrogen gas production greatly enhanced using an algal-bacterial co-culture system, under conditions of sulfur deprivation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-34. (canceled)

35. A method of generating hydrogen gas, the method comprising sequentially
(a) propagating photosynthetic algae in a propagation medium, said propagation medium comprising sulfur;
(b) culturing said algae in a culturing medium which comprises a reduced amount of sulfur compared to said propagation medium, for a length of time sufficient to establish micro-oxic culturing conditions, wherein said culturing is co-culture with added bacteria for at least a portion of said length of time;
(c) culturing said algae in said culturing medium under micro-oxic culturing conditions, thereby generating the hydrogen gas; and
(d) collecting said hydrogen gas,
wherein said length of time to micro-oxic culture conditions of step (b) is reduced compared to the length of time of a similar culture of algae not co-cultured with added bacteria.

36. The method of claim 35, wherein said length of time of step (b) is until oxygen consumption of said algal culture is equal to or greater than photosynthetic oxygen production of said algal culture, as measured under high intensity illumination.

37. The method of claim 35, further comprising depleting at least some of said bacteria in said culturing medium to generate a bacteria-reduced culturing medium following step (b) and prior to, or during step (c).

38. The method of claim 37, wherein said depleting is effected wherein oxygen consumption of said algal culture is equal to or greater than photosynthetic oxygen production of said algal culture, as measured under high intensity illumination.

39. The method of claim 37, wherein said bacteria-reduced culturing medium is essentially devoid of said bacteria.

40. The method of claim 35, wherein said propagation medium is essentially devoid of said bacteria.

41. The method of claim 35, wherein said culturing medium is essentially devoid of sulfur.

42. The method of claim 35, wherein said culturing said algae under micro-oxic conditions is effected under illuminated conditions.

43. The method of claim 35, wherein said co-culture is effected under illuminated conditions.

44. The method of claim 35, wherein said bacteria are comprised in a bacterial containment in fluid association with an algae containment, said algae containment separated from said bacterial containment by a fluid- and gas-permeable and bacterial impermeable barrier.

45. The method of claim 44, wherein said bacterial containment is located within said algae containment and separated therefrom by said fluid- and gas-permeable and bacterial impermeable barrier.

46. The method of claim 35, wherein said culturing in (b) and (c) is for about 4 to about 60 hours.

47. The method of claim 35, wherein said algae comprises green algae.

48. The method of claim 35 wherein said algae comprises unicellular, photosynthetic algae.

49. The method of claim 35, wherein said algae comprise algae having a Fe-hydrogenase enzyme.

50. The method of claim 35, wherein said algae is selected from the group consisting of Platymonas subcordiformis, Rhodobacter sphaeroide and Chlamydomonas reinhardtii.

51. The method of claim 35, wherein said bacteria comprises oxygen-consuming bacteria.

52. The method of claim 35, wherein said bacterium is Pseudomonas fluorescens.

53. A method of generating hydrogen gas, the method comprising sequentially
(a) propagating photosynthetic algae in a propagation medium, said propagation medium comprising sulfur;
(b) co-culturing said algae with bacteria in a culturing medium for a length of time sufficient to ensure reduced oxygen culturing conditions, wherein said culturing medium comprises a reduced amount of sulfur compared to said propagation medium;
(c) depleting at least some of said bacteria in said culturing medium to generate a bacteria-reduced culturing medium;
(d) culturing said algae in said culturing medium for a length of time sufficient to ensure micro-oxic culturing conditions;
(e) culturing said algae in said culturing medium under anaerobic culturing conditions, thereby generating the hydrogen gas; and
(f) collecting said hydrogen gas.

54. The method of claim 53, wherein said length of time of step (d) is until oxygen consumption of said algal culture is equal to or greater than photosynthetic oxygen production of said algal culture, as measured under high intensity illumination.

55. The method of claim 53, wherein said algae comprise unicellular, photosynthetic algae having a Fe-hydrogenase enzyme.

56. The method of claim 53, wherein said bacteria comprises an obligatory aerobic bacteria.

57. The method of claim 53, wherein said bacterium is Pseudomonas fluorescens.

58. A system for generating hydrogen gas, the system comprising:
(a) a sealed culture vessel comprising photosynthetic algae and bacteria co-cultured in a culturing medium comprising a reduced amount of sulfur as compared to an algal propagation medium;
(b) a source of illumination of said culture vessel; and
(c) a means for collecting hydrogen gas from said culture vessel,
wherein said bacteria are comprised in a bacterial containment in fluid association with an algae containment, said algae containment separated therefrom by a fluid- and gas-permeable and bacterial impermeable barrier.

59. The system of claim 58, wherein said bacterial containment is located within said algae containment and separated therefrom by said fluid- and gas-permeable and bacterial impermeable barrier.
